(19) United States
(12) Patent Application Publication
(10) Pub. No.: US 2005/0022815 A1
(43) Pub. Date: Feb. 3, 2005

(54) Apparatus and method for monitoring supplemental oxygen usage

(75) Inventor: Frank R. Frola, Somerset, PA (US)

Correspondence Address: MACMILLAN SOBANSKI & TODD, LLC;ONE MARITIME PLAZA FOURTH FLOOR, 720 WATER STREET, TOLEDO, OH, 43604-1619, US

(73) Assignee: Sunrise Medical HHG Inc., Longmont, CO (US)

(21) Appl. No.: 10/630,021

(22) Filed: Jul. 30, 2003

Related U.S. Application Data
(60) Provisional application No. 60/482,356, filed on Jun. 25, 2003.

(51) Int. Cl. A61M 16/00; A62B 7/00

(52) U.S. Cl. 128/204.21; 128/205.22

(57) ABSTRACT

A device for monitoring and recording the usage of gas supply apparatus. The device includes erasable memory for storing the usage data and a connection for periodically downloading the data to a personal computer.

### CROSS REFERENCE TO RELATED APPLICATIONS

This application claims the benefit of Provisional Patent Application Ser. No. 60/482,356 filed Jun. 25, 2003.

### BACKGROUND OF INVENTION

This invention relates in general to devices for mounting upon a compressed oxygen cylinder or liquid oxygen reservoir for controlling the delivery of supplemental oxygen to an ambulatory patient and in particular to an apparatus and method for monitoring the operation of the device.

As the number of aged people in the population increases, there is an increasing number of people who require supplemental oxygen therapy. Many of these people are ambulatory and are capable of leaving the home and hospital. However, they require a portable source of supplemental oxygen in order to remain mobile. In the most basic supplemental oxygen system, compressed oxygen from a tank is supplied to the ambulatory patient through a pressure reducing regulator and a tube connected to a nasal cannula. The difficulty with the basic system is that the oxygen flow must be continuous. This results in an unnecessarily high oxygen consumption. Either the mobile time is severely limited or the patient must carry or push a heavy large capacity oxygen cylinder. The wasted oxygen also increases the expense of oxygen therapy.

Since the normal breathing pattern is to inhale about one-third of the time and to exhale and pause about two-thirds of the time, the constant flow gas delivery devices waste more than two-thirds of the oxygen since the oxygen is delivered to the patient during the exhalation and pause portion of the breathing cycle in addition to the inhalation portion of the cycle. In addition, it has been recognized that a patient's airway includes significant dead air space between the mouth and nose and the oxygen adsorbing portions of the lungs. Only oxygen in the portion of the respiratory gas which reaches the alveoli is absorbed. This oxygen is in the leading portion of the flow of respiratory gas when the patient initially begins to inhale. One recent trend in the design of portable respiratory oxygen management systems is a pulse-type flow controller which delivers a fixed volume or bolus of the respiratory gas only at the initiation of a patient's inhalation cycle. The gas savings permits smaller and lighter portable oxygen systems with increased operating time. An exemplary prior art oxygen flow controller is shown, for example, in U.S. Pat. No. 4,461,293.

The pulse-type gas flow controllers typically use a sensor to determine when the initial point of inhalation occurs. Upon sensing the initiation of inhalation, the device opens a valve to deliver a short, measured dose of oxygen at the leading edge of the inhalation cycle. Since all of this dose finds its way deep into the lungs, less oxygen is required to accomplish the same effect than with the more wasteful continuous flow delivery method. Therefore, with the pulsed delivery method, the respiratory gas supply is conserved while still providing the same therapeutic effect. Typically, an oxygen supply with a pulse flow controller will last two to four times longer than a similarly sized continuous flow oxygen supply. However, the actual oxygen usage will vary depending upon the particular user and the user's activity level. Since the oxygen usage directly affects the frequency of gas cylinder replacement, it would be desirable to monitor the actual usage of the gas supplied by system.

### SUMMARY OF INVENTION

This invention relates to an apparatus and method for monitoring the operation of a device devices for controlling the delivery of supplemental oxygen to an ambulatory.

The present invention contemplates a device adapted to be connected to controller for a gas supply apparatus and at least one storage device connected to said gas usage monitor that is operable able to store said gas usage data. In the preferred embodiment, the device is a microprocessor and the storage device includes at least one electrically erasable programmable read-only memory chip. The device also includes a connector for downloading the stored data to an external device, such as a personal computer.

The present invention also contemplates a method for monitoring a gas supply apparatus comprising the steps of providing a gas usage monitor having at least one storage device. The usage monitor monitors and records gas usage in the storage device. The stored data is then periodically downloaded to an external device, such as a personal computer.

Various objects and advantages of this invention will become apparent to those skilled in the art from the following detailed description of the preferred embodiment, when read in light of the accompanying drawings.

### BRIEF DESCRIPTION OF DRAWINGS

FIG. 1 is a front perspective view of a compressed gas cylinder fitted with a gas management device that includes a capability to monitor gas usage in accordance with the present invention.

FIG. 2 is schematic diagram of a control circuit for the gas management system shown in FIG. 1.

FIG. 3 is a flow chart for a method for monitoring the gas usage of the gas management system shown in FIG. 1.

### DETAILED DESCRIPTION

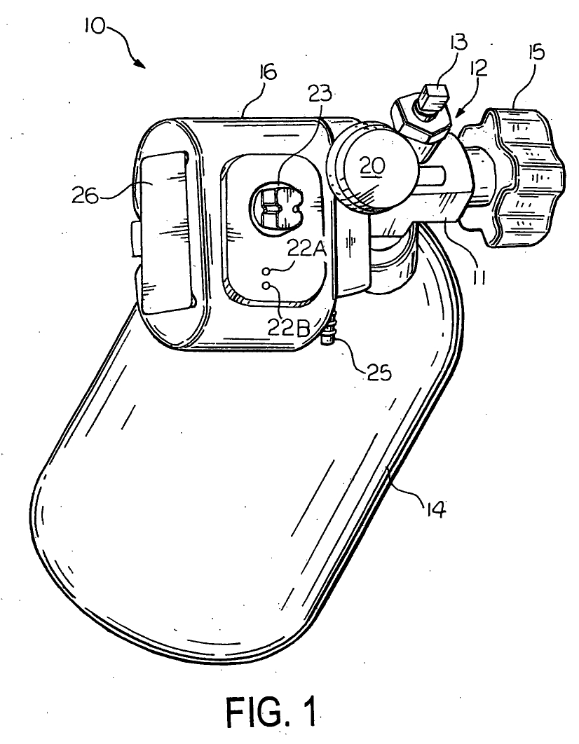

Referring now to the drawings, FIG. 1 shows a gas management device 10 in accordance with the present invention. The gas management device 10 has a regulator base 11 with a center opening 12 which is configured to fit over a post 13 of an gas cylinder 14. In the preferred embodiment, the cylinder 14 contains pressurized oxygen; however, the cylinder 14 also may be a liquid oxygen reservoir. A round handle 15 projects from the regulator base 11 and may be manually rotated for releasably securing the device 10 to the gas cylinder 14. It will be appreciated that other handle shapes also may be provided.

The components of the gas management device 10 are contained within a housing 16. As shown in FIG. 1, several indicating devices are located upon the surface of the housing 16. These may include a pressure gauge 20 and a pair of LED indicators 22A and 22B. The indicating LEDs 22A and 22B will be discussed in detail below. In addition, a mode selection switch 23 for selecting between a plurality of maximum pulse dose flow rates or continuous flow is mounted upon the housing 16. The selection switch 23 provides a continuous-type oxygen delivery mode, should the user require a continuous dose from his portable device. This also permits continuation of oxygen therapy in the event that the pulse flow controller should fail. Additionally, the selection switch 23 has an off position to shut down the device 10. A barbed fitting 25 projects from the housing 16 for connection to a tube (not shown) which in turn connects to a conventional nasal cannula (not shown) for delivering the oxygen to the user. An access door 26 is located on a side of the housing 16 for replacing a battery, or batteries, which power the device 10.

The housing 16 is preferably molded from a lightweight and durable material, such as a plastic. It is preferred that the material used for the housing 16 also have flame retardant characteristics since it may be exposed to high oxygen concentration gas. One suitable material which meets these criteria is an ABS such as Cycolac KJW manufactured by General Electric Company. ABS is the material of choice because of its flame retardancy and excellent impact properties. Additional details of a similar gas management device 10 are included in U.S. Pat. No. 5,755,224, which is incorporated herein by reference.

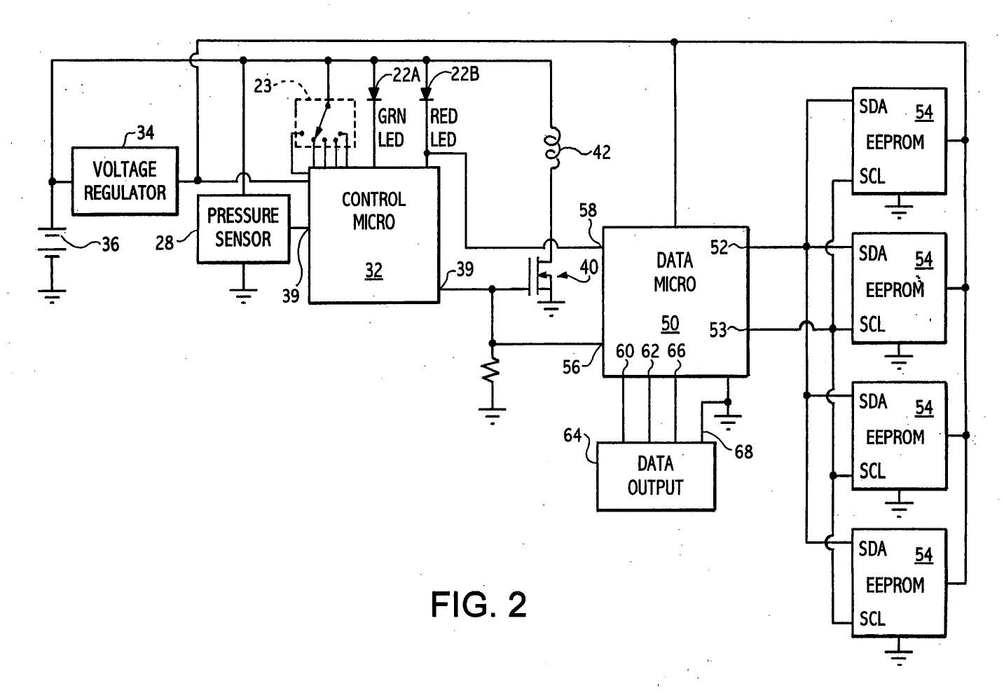

FIG. 2 is a schematic diagram of the control circuit 30 for the gas management device 10 that includes a capability to monitor gas usage that is in accordance with the present invention. The control circuit 30 is mounted upon a circuit substrate (not shown) that is entirely enclosed within the housing 16. The circuit 30 includes a control microprocessor 32 that is programmed to operate the gas management device 10. As will be described below, the control microprocessor 32 is responsive to a pressure sensor signal to open a normally closed oxygen supply solenoid valve (not shown) and deliver a bolus of oxygen to the user. In the preferred embodiment, the control microprocessor 32 is a Texas Instrument MSP430 microchip; however, other microprocessors also may be used. The microprocessor 32 is supplied power through a conventional voltage protection circuit 34 from a voltage supply 36. In the preferred embodiment, the voltage supply 36 is a pair of AA batteries connected in series that have a voltage output in the range of 1.8 to 3.2 volts. The voltage protection circuit 34 is operative to maintain the input voltage level to the microprocessor 32 by preventing the voltage from being pulled down when the solenoid valve for supplying oxygen to the user is actuated. The microprocessor 32 is operative to monitor the output voltage from the voltage protection circuit 34 to determine if the batteries are approaching the end of their useful life. The control microprocessor 32 also is electrically connected to a pressure sensor 38. The pressure sensor 38 is mounted upon the circuit substrate and communicates with a port formed in a manifold block (not shown) disposed within the housing 16. The pressure sensor 38 detects a reduced oxygen pressure within the manifold block when the user inhales and generates an electrical signal indicative thereof that is applied to a pressure signal port 38A of the control microprocessor 32.

The microprocessor 32 has an output signal port 39 that is electrically connected to the gate of a Field Effect Transistor (FET) 40. The FET 40 is electrically connected between ground and one end of a solenoid coil 42 for the oxygen supply valve. The other end of the solenoid coil 42 is connected directly to the voltage supply 36. Upon receiving a signal from the pressure sensor 38 that the user is inhaling, the microprocessor 32 is operative to cause the FET 40 to apply a voltage to the FET gate. The voltage on the FET gate switches the FET 40 to a conducting state and thereby energizes the solenoid coil 42. Upon energization of the solenoid coil 42, the associated normally closed oxygen supply valve opens to supply pressurized oxygen to the user. When the user stops inhaling, the pressure transducer 38 reverts to its original state which, in turn, causes the microprocessor 32 to remove the voltage applied to the FET gate, returning the FET 40 to an non-conducting state. When the FET 40 returns to the non-conducting state, the solenoid coil 42 is de-energized, allowing the oxygen supply valve to return to a closed position and thereby cutting off the flow of pressurized oxygen to the user. Alternately, depending upon the position of the mode selection switch 23, the solenoid valve may be closed after the elapse of a predetermined time that corresponds to the selected dose flow rate.

As shown in FIG. 2, the indicator LED's 22A and 22B are connected directly between the voltage supply 36 and the control microprocessor 32. The LED's 22A and 22B provide indications of both operation of the device 10 and battery status to the user. In the preferred embodiment, the left LED 22A is red while the right LED 22B is red. The green LED 22A is illuminated by the control microprocessor 32 while the solenoid valve is open, that is when the user is inhaling, and the battery 36 has sufficient energy to operate the device 10. Upon detecting that the output voltage from the batteries has dropped below a predetermined threshold, which is an indication that the batteries are beginning to reach the end of their useful life, the microprocessor 32 switches to the red LED 22B. As before, the red LED 22B is illuminated by the control microprocessor 32 while the solenoid valve is open, but the color warns the user that he should replace the batteries. Thus, the LED's 22A and 22B provide a visual indication to the user that the device 10 is operative and also provide a visual warning when the batteries need renewal. The voltage threshold for switching from the green LED 22A to the red LED 22B is selected to provide a warning sufficiently in advance of the batteries actually becoming exhausted to allow the user adequate time to replace the batteries. A multiple position mode selection switch 23 also is connected between the voltage supply 36 and the control microprocessor 32.

The control microprocessor 32 has a standby mode in which the microprocessor's oscillator is turned off to conserve battery life. If the microprocessor 32 does not receive an inhalation signal for a predetermined amount of time, the microprocessor 32 enters a “sleep” mode with most of its functions shut off. In the preferred embodiment, the time period is selected as one minute. However, upon receiving a signal from the pressure transducer 38 that the user has drawn a breath, the microprocessor 32 will awaken by restarting the oscillator and provide oxygen in accordance with the setting of the mode selection switch 23.

The control circuit 10 also includes a second data microprocessor 50 that is operative to collect operational data for the device 10. In the preferred embodiment, a PIC 18F627 available from Microchip Technology Inc. is used; however, other microprocessors also may be used. The data microprocessor 50 has a data output port 52 that is connected to the serial data acquisition port of each of a plurality of Electrically Erasable Programmable Read-Only Memory (EEPROM) chips 54 by a Serial Data Acquisition Line (SDA). The EEPROM chips 54 both read and write data. In the preferred embodiment, each of the EEPROM chips 54 are a 24LC256 chip, also available from Microchip Technology Inc.; however, other memory chips also may be used. While four EEPROM chips 54 are shown in FIG. 2, it will be appreciated that the invention also may be practiced with more or less chips than shown. A clock output port 53 on the data microcomputer 50 is connected to a clock input port on each of the EEPROM chips 54 by a Serial Clock Line (SCL) and supplies clock signals for synchronizing the operation of the data microprocessor 50 and the EEPROM chips 54. The serial data acquisition and serial clock lines define an Inter-Integrated Circuit bus, or I2C bus, for communication between the data microprocessor 50 and the EEPROM chips 54. The I2C bus allows communication between the microprocessor 50 and multiple memory chips 54 over only two wires.

Both the data microprocessor 50 and the EEPROM chips 54 are electrically connected to the output of the voltage protection circuit 34. The data microprocessor 50 has a first data input port 56 that is connected to the gate of the FET 40. Additionally, a second data input port 58 of the data microprocessor 50 is connected to the cathode of the red LED 22B. During normal usage, the data microprocessor 50 receives device usage data at the first data input port 56. However, upon the battery voltage falling below the voltage threshold described above, the data microprocessor 50 will begin receiving device usage data at the second data input port 58. The change of data input ports functions as a low voltage/battery failure signal to the data microprocessor 50. The data microprocessor 50 is responsive to the low voltage/battery failure signal to stop operating and thereby conserve battery life. As will be described below, the data microprocessor 52 operates only during inhalation by the user to further conserve the batteries.

The data microprocessor 50 includes a pair of data output ports 60 and 62 that are connected to a data output interface 64. As shown in FIG. 2, the output interface 64 also includes a power input port 66 and a ground connection 68. In the preferred embodiment, the data output interface 64 includes an electrical connector for connecting an external personal computer (not shown) to the data microprocessor 50 for downloading of stored usage data. Also in the preferred embodiment, access to the data output interface 64 by the device user is limited by locating the interface 64 behind the batteries (not shown). Thus, the batteries must be removed prior to downloading the stored data. However, the invention also may be practiced with the data output interface 64 positioned with connector accessible from outside the device 10. When connected, the external personal computer provides power to the EEPROM chips 54 across the power input port 66 and ground 68. The external personal computer also would access the I2C bus through the data output ports 60 and 62 for reading and clearing the data stored on the EEPROM chips 54. The preferred embodiment also contemplates that the external connector that connects to the output interface 64 includes a circuit (not shown) to convert the TTL levels from the EEPROM chips 54 into a RS32 signal for use by the personal computer. Additionally, the output interface 64 may include more contact points than are shown in FIG. 2.

The operation of the data monitoring portion of the device 10 will now be described. In the preferred embodiment, the data microprocessor 50 monitors the input ports 56 and 58 to count the number of breaths taken by the user during a predetermined time period. Again, for the preferred embodiment, the predetermined time period is one minute. Additionally, the duration of the last breath during the time period is measured and the breath duration is used as an average breath duration during the minute. At the end of the predetermined time period, two bytes, representing the number of breaths and breath duration, are serially written to one of the EEPROM chips 54, where the data is stored. The number of breaths and breath duration are coded into 1-8 bits in each byte. If the number of breaths is zero, the duration will also be zero. In this case only one byte would be needed for data; however, the use of one byte would make tracking of the records difficult. Accordingly, when there are no breaths, the microprocessor simply downloads a double zero for zero usage and then advances to the next data storage address. For the circuit 30 shown, it expected that the four EEPROM chips 54 can store 44.5 days of data.

Periodically, the stored data is downloaded into an external personal computer. As the data is downloaded, the EEPROM chips 54 are erased. Thus, upon completion of the data download, the usage monitoring can resume. In the preferred embodiment, the data is downloaded once per month. The personal computer has software for manipulating the downloaded data to produce a device activity and oxygen usage report. In the preferred embodiment, the report can provide hourly and/or daily usage data.

Similar to the control microprocessor 32, the data microprocessor 50 also has a standby mode in which the microprocessor's oscillator is turned off to reduce power use. An external event such as the valve drive line going high re-starts the oscillator and triggers an interrupt in the microprocessor 50 so that the microprocessor only has to be active while it is processing the signal. Another event that can wake the data microprocessor 50 from standby is an internal signal from a counter connected to a 32.768 khz crystal (not shown) that wakes the microprocessor every 4 seconds to keep track of the passage of real time. After every 15 counter events, one minute has elapsed and the accumulated breath and valve on time data is stored to the EEPROM chips 54. The breath and valve on time variables are then cleared for use in accumulating data during the next minute.

While the preferred embodiment has been described as counting the number of breaths each time period and measuring the last breath during each minute, it will be appreciated that the invention also may be practiced to accumulate other data. For example, the duration of each breath during the time period can be measured and the durations averaged. Alternately, the duration of each breath can be measured and then saved; however, the additional storage required to do so will decrease the total time between data downloads. Also, the number of breaths can be counted for a different time period, such as, for example, five or ten minute intervals. Additionally, the EEPROM chips 54 can be configured to construct multiple data tables. The EEPROM chips 54 would then record the number of breaths for each of the flow settings, pulsed or continuous flow mode, in one table and the number of minutes of use at each flow setting in the another table.

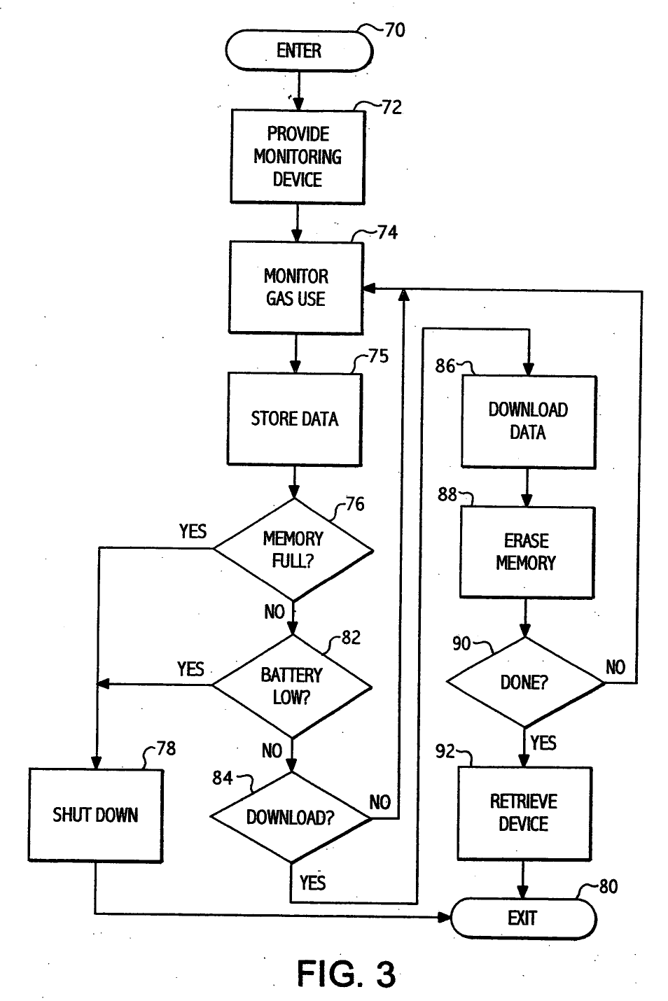

A flow chart for an algorithm for implementing the above operation is shown in FIG. 3. The algorithm is entered through block 70 and proceeds to functional block 72 where the monitoring device described above is provided by an organization that desires to gather usage data. The algorithm then continues to functional block 74 where the usage of the pressurized gas supply is monitored for a predetermined period of time. As described above, in the preferred embodiment, the number of breaths, as determined from valve on time, during one minute are counted and the duration of one of the breaths during the minute is measured. In order to conserve battery life, the monitoring device is in a standby mode until activated by detection of a user breath. Additionally, in the preferred embodiment, the monitoring device will enter the standby mode between breaths. Thus, the device is active for only about a third of the time that it is in use. The data is stored in a reusable memory chip in functional block 75. The algorithm then advances to decision block 76.

In the preferred embodiment, the monitoring device will operate until the memory is full, the batteries are depleted or the data is downloaded. Accordingly, in decision block 76, the algorithm determines whether the memory is full. If the memory is full, the algorithm transfers to functional block 78 where the monitoring device is de-activated pending a memory download and then exits through box 80. If the memory is not full, the algorithm transfers to decision block 82.

In decision block 82, the monitoring device determines whether the battery is low. If the battery is low, the algorithm transfers to functional block 78 where the monitoring device is de-activated pending a memory download and then exits through box 80. If the memory is not full, the algorithm transfers to decision block 84.

In decision block 84, the algorithm determines whether a download condition exists. In the preferred embodiment, the download condition is determined when an external connector is attached to the monitoring device for downloading the data. If, in decision block 84 it is determined that a download condition does not exist, the algorithm returns to functional block 74 and continues to monitor the gas usage. If, in decision block 84 it is determined that a download condition does exist, the algorithm advances to functional block 86.

In functional block 86, the stored data is downloaded into an external personal computer. The personal computer utilizes the data to generate a gas usage report. In the preferred embodiment an Excel™ Computer Program is used to generate the report. Upon completion of the data download, the algorithm continues to functional block 88 where the memory is erased. Alternately, the memory can be erased concurrently with the data download in functional block 86. The algorithm then advances to decision block 90.

In decision block 90, the organization desiring the data determines whether the monitoring is completed. If the monitoring is completed, the algorithm advances to block 92 where the monitoring device is retrieved and then exits trough block 80. If the monitoring is not completed, the algorithm returns to functional block 74 and continues to monitor gas usage.

In addition to the discontinuance of monitoring upon the memory being full or the battery voltage low, the invention also contemplates that the monitoring may be discontinued upon lapse of a predetermined time period. If this option is included, an additional decision block (not shown) would be included after decision block 82 in FIG. 3 in which the total elapsed monitoring time is compared to a predetermined monitoring duration period. Upon the total elapsed monitoring time being greater than or equal to the monitoring duration period, the algorithm would transfer to functional block 78 where the monitoring device is de-activated. If the total elapsed monitoring time being less than the monitoring duration period, the algorithm would continue to decision block 84.

While the preferred embodiment has been illustrated and described for delivery of oxygen, it will be appreciated that the invention also may be practiced for delivery of other gases. Also, while two microprocessors 32 and 50 were shown in FIG. 2, it will be appreciated that the invention also may be practiced with the functions of the two microprocessors combined in a single microprocessor (not shown). Similarly, the invention also may be practiced by using an Application Specific Integrated Circuit (ASIC) in place of the data microprocessor 50. Additionally, the invention also may be practiced to monitor gas usage of a Continuous Positive Airway Pressure (CPAP) apparatus, such as the device described in U.S. Pat. No. 5,551,419.

The principle and mode of operation of this invention have been explained and illustrated in its preferred embodiment. However, it must be understood that this invention may be practiced otherwise than as specifically explained and illustrated without departing from its spirit or scope.

1. An apparatus for recording usage of a gas supply apparatus comprising:
a device adapted to be connected to the gas supply apparatus controller, said device operable to monitor gas usage data; and
at least one data storage device connected to said gas usage monitor, said storage device operable to store said gas usage data.

2. The apparatus according to claim 1 wherein said gas usage monitor includes a microprocessor and further wherein said data storage device includes at least one electrically erasable programmable read-only memory chip.

3. The apparatus according to claim 2 wherein said gas usage data includes the number of operations of the gas supply apparatus during a predetermined time period and the duration of one said operations during said predetermined time period.

4. The apparatus according to claim 3 further including a data interface connected to said microprocessor, said data interface adapted to be connected to a personal computer and operative to download the data stored in said electrically erasable programmable read-only memory chip to said personal computer.

5. The apparatus according to claim 4 wherein said data personal computer is also operative to erase said electrically erasable programmable read-only memory chip as the data is downloaded.

6. The apparatus according to claim 5 wherein said microprocessor includes a sleep mode such that said microprocessor is only active during data collection, said sleep mode being interrupted upon the gas supply apparatus user drawing a breath.

7. The apparatus according to claim 5 wherein said data storage device includes a plurality of electrically erasable programmable read-only memory chips and said electrically erasable programmable read-only memory chips are connected to said microprocessor with a serial data bus.

8. The apparatus according to claim 5 wherein said microprocessor is a data microprocessor and further wherein the gas supply apparatus includes a control microprocessor that is connected to a solenoid valve that is operative to control the supply of gas from the apparatus to a user, said control microprocessor also being connected to said solenoid valve to receive operating data.

9. The apparatus according to claim 5 wherein said microprocessor is also connected to a solenoid valve and operable to control the supply of gas from the apparatus to a user.

10. The apparatus according to claim 7 wherein said microprocessor is responsive to a low voltage condition to cease operation whereby battery life is extended.

11. The apparatus according to claim 5 wherein said data includes the number of breaths taken by user during a predetermined time period and an average breath duration for said time period.

12. The apparatus according to claim 11 wherein said predetermined time period is one minute.

13. The apparatus according to claim 11 wherein the duration of the last breath during said predetermined time period is used as said average breath duration.

14. The apparatus according to claim 4 wherein said gas supply includes a cylinder of pressurized oxygen.

15. The apparatus according to claim 4 wherein said gas supply includes a liquid oxygen reservoir.

16. A method for monitoring the usage of a gas supply apparatus comprising the steps of:
(a) providing a device adapted to be connected to the gas supply apparatus controller, the device operable to monitor gas usage data and at least one storage device connected to said gas usage monitor, the storage device operable to store the gas usage data;
(b) monitoring the usage of the pressurized gas supply apparatus controller;
(c) periodically downloading the data to an external personal computer; and
(d) erasing the storage device.

17. The method according to claim 16 wherein step (b) is discontinued following the elapse of a predetermined time period.

18. The method according to claim 16 wherein said gas usage monitor enters a sleep mode between user breaths.

19. The method according to claim 16 wherein the data includes the number of breaths taken by a user during a predetermined time period and an average breath duration for said time period.

20. The method according to claim 16 wherein the gas supply provided in step (a) includes a cylinder of pressurized oxygen.
